(19) United States
(12) Patent Application Publication
(10) Pub. No.: US 2007/0093687 A1
(43) Pub. Date: Apr. 26, 2007

(54) Wearable penile lengthening vacuum device, particularly for use with standard condoms

(76) Inventor: Juergen Hoefer, Hamburg (DE)

Correspondence Address: Mr. Juergen Hoefer, Grindelallee 91, Hamburg, D-20146, DE

(21) Appl. No.: 11/373,201

(22) Filed: Mar. 13, 2006

(30) Foreign Application Priority Data
Oct. 26, 2005 (DE) DE102005051528.2
Nov. 26, 2005 (EP) EP05025858

(51) Int. Cl. A61F 5/00; A61F 6/04

(52) U.S. Cl. 600/41; 128/844

(57) ABSTRACT

A wearable penile lengthening vacuum device, comprising a hollow body (2), which enables the insertion of a penis (1) without any problem, whereby both a part of the outer surface of a hollow body (2) and the proximal part (1a) of a penis protruding from the hollow body (2) are wrapped in an airtight manner by the same highly elastic jacket (14) (a standard condom (5) or a silicone penis sleeve (6)), which thereby connects the penis (1) and the hollow body (2). The user can create a vacuum (lowered pressure) in the hollow body (2) by introducing a tractive force on a pulling means (7) fixed to the sealed hollow body (2). An extension of the penis (1) will be caused by the continuous stretching effect of a continuously exerted tractive force, as a large part of the tractive force is (due to the vacuum resulting from this traction force) transformed into a suction force, as the exterior air with atmospheric pressure is prevented from penetrating into the hollow body (2). Said suction force is evenly distributed on the tissue of the penis (1) surrounded by the vacuum.

### BACKGROUND OF THE INVENTION

1. Field of the Invention

The present invention relates to a wearable penile lengthening vacuum device, especially for use with standard condoms, and more particularly to a wearable penile lengthening vacuum device, which enables the unproblematic insertion and vacuum assisted stretching of a penis in a hollow body. The device creates the vacuum (without the help of a pump) exclusively by introducing a tractive force on pulling means fixed to the hollow body, which can be sealed by either an inexpensive standard condom or a silicon sleeve.

2. Related Art

Various devices for penis extension are known, whereby the effect of the devices suggested in U.S. Pat. No. 4,753,227, U.S. Pat. No. 5,306,227, U.S. Pat. No. 5,836,864 and U.S. Pat. No. 6,926,666 is caused by the creation of a vacuum. This invention is in contrast to said devices for penis extension using a vacuum, as it is necessary to use pumps for the creation of a vacuum in these devices, which results in them being unwieldy and complicated to use. Furthermore—in contrast to this invention—these devices cannot be discreetly worn under normal clothing. Therefore the use of these devices is limited to the private sphere of the user. Only a relatively short daily period of use is therefore possible, which in turn results in an unsatisfactory success for the user.

Finally a device for penile tension is sold by Messrs. Phalomed Manufacturing Ltd., Malta, under the trade name “Phallosanu”. This device exerts a vacuum when a pulling force is introduced to a special stretch condom. The principle of the “Phallosan” is shown in disclosure document DE10001331A1. This invention is also in contrast to both the “Phallosan” and a condom-like little hat as suggested in DE10001331A, which lies close to the glans in a tight-fitting manner. As the annular section of the little hat lies close to the penis shaft immediately behind the glans, a lowered pressure is created in the hat as a result of the introduction of a stretching force on a stretching element. A slightly changed device is sold under the trade name “Phallosan”. The “Phallosan” has a number of grave deficiencies. In the following only the most important deficiencies are mentioned:

In case of the “Phallosan”:
1. both the proximal and the distal end of the used special condom for continuous stretching must be sealed absolutely airtight, otherwise this condom does not work.
2. a very expensive special condom for continuous stretching is used. This extremely thick-walled special condom looks like an urinal condom. At its tip is a rubber tube, which also has extremely thick walls. This is necessary to enable that a tractive force can be exerted on the rubber tube.
3. the penis is not visible during the treatment, so that an inspection of same during the treatment is not possible.
4. in order to obtain an absolutely airtight seal, the end of the rubber tube must be pushed exactly into the middle of a special clip, which requires considerable force and manual dexterity.
5. only glans and foreskin are exposed to the suction effect.
6. glans and foreskin must be forced into the bell-shaped extension of the special condom. This requires a problematic and somewhat unhygienic procedure, as glans and foreskin must be moistened with saliva.
7. the special condom is inevitably wetted, when the user is urinating.
8. When the very thick-walled rubber skin is rolled back, a sticking effect easily occurs. This makes the usage increasingly difficult and later even impossible.

In case of this invention:
1. the problem of the distal air-tight closing does not exist because the hollow body (2) has either no distal opening at all or an opening with an absolutely air-tight closure (10).
2. a normal standard condom (5) or a penis sleeve (6) (both customary in trade) can be used, so that the treatment is not expensive. Furthermore the pulling means (7) is not fixed to the condom, but to the hollow body (2).
3. using a transparent hollow body the penis is visible during treatment, so that an inspection of same during the treatment is possible.
4. no special dip but only a simple connecting part, such as for example a (spring) hook is required.
5. the vacuum surrounds not only glans and foreskin but also a part of the penis shaft, so that also the penis shaft is directly stretched by the vacuum.
6. there is no difficulty at all in the introduction of glans, foreskin and penis shaft into the hollow body (2) and no need for force when these penis parts are inserted.
7. the user can pass water through the opening of the closure (10), so that hollow body (2) and jacket (14) are not wetted.
8. normal standard condoms or alternatively penis sleeves (both customary in trade) can be employed. The use of same does not cause the problematic sticking of very thick-walled rubber skin.

For more details please see the instructions of use of the “Phallosan”.

### SUMMARY OF THE INVENTION

This invention results from the grave deficiencies of the existing penis extension devices of the same family. The task is the creation of a penile lengthening vacuum device, particularly for use with inexpensive standard condoms, with the above-mentioned advantages, which can be discreetly worn. This invention tries to find a way to eliminate the suffering by—either subjectively felt or really existing—hypoplasia of the penis by a totally painless and cost-effective extension (thickening and lengthening) of the penis. The task of the invention is to create a device, which can a) provide unproblematic and cost-effective help to patients with prostate carcinoma after a postoperative penis retraction (caused by the withdrawal of hormones), and which is b) also especially suited for those paraplegics who show the phenomenon of the “retractio penis” (retraction of the penis into the subcutaneous fatty tissue) as a result of the paralysis of the abdominal muscles and the sedentary position in the wheel-chair. The technique here presented makes it possible to pull the micro-penis created by the retraction out of the fatty tissue, and to achieve at the same time an objective lengthening and thickening of the complete penis (and not only of the glans).

This invention describes a wearable penile lengthening vacuum device, particularly for use with a standard condom, comprising a hollow body (2), which enables the insertion of a penis (1) without any problem, whereby both a part of the outer surface of a hollow body (2) and the proximal part (1a) of a penis protruding from the hollow body (2) are wrapped in an airtight manner by the same highly elastic jacket (14) (a standard condom (5) or a silicone penis sleeve (6)), which thereby connects the penis (1) and the hollow body (2). Said jacket (14) is sized for tightly wrapping said proximal part (1a) of the penis and said outer surface of the hollow body (2), and has an elasticity sufficient for the airtight or almost airtight sealing of the inner cavity of the hollow body (2).

The user can create a vacuum (lowered pressure) in the hollow body (2) by introducing a tractive force on a pulling means (7) fixed to the sealed hollow body (2). A large part of the tractive force is (due to the vacuum resulting from this traction force) transformed into a suction force, as the exterior air with atmospheric pressure is prevented from penetrating into the sealed hollow body (2). Said suction force is evenly distributed on the tissue of the penis (1) surrounded by the vacuum. A durable extension of the penis (1) will be caused by the continuous stretching effect of a continuously exerted tractive force.

The interior of said hollow body (2) forms a hollow space (3) (a cavity) that is suitable for the admission of a penis. The hollow body (2) has an opening only at its proximal end. This proximal open end (2a) has an airtight seal. The integrity of this airtight seal is guaranteed by a jacket (14) (preferably a simple standard condom (5) or a silicone penis sleeve (6) (both customary in trade)), which has two open ends. As condoms do of course only have one opening at their proximal end, it is necessary to cut off the tip (5a) of the condom (5). This is not necessary if silicone penis sleeves are used, as these sleeves have two open ends anyhow. If the jacket (14) is a condom (5), same must at first be unrolled over the outer surface (4) of the hollow body (2) and then over the proximal part (1a) of the penis. In every case the hollow body (2) (into which the penis (1) is introduced) has an outer diameter that exceeds that of the penis. As a result, the compressive force of the jacket (14) is so great that an absolutely airtight seal is normally achieved between the inner surface (8) of the jacket (14) and the outer surface (4) of the hollow body (2). As a result the exterior air with atmospheric pressure (9) cannot penetrate (between inner surface (8) of the jacket (14) and outer surface (4) of the hollow body (2)) into the inner cavity (3) of the hollow body (2).

The highly elastic jacket (14) produces (as a result of its compressive action) normally an airtight seal of the cavity (3) of the hollow body (2), as the proximal part of the same jacket (14) presses on the skin of the proximal part (1a) of the penis (1) adjacent to the proximal open end (2a) of the hollow body (2). Due to this envelopment, the penetration of the exterior air with atmospheric pressure (9) into the inner space (cavity) (3) of the hollow body (2) (along the skin of the proximal part (1a) of the penis (1)) is prevented. Although by this way the necessary airtight seal of the inner cavity (3) of the hollow body (2) has already been achieved, the pressing of the condom (5) against the skin of the proximal part (1a) of the penis can still be supported by other sealing means, such as for example at least one piece of Latex band (11), any kind of rubber band, Velcro band or silicone sleeve, so that an airtight seal can be guaranteed also for a longer period. Also in case only an almost airtight seal can be achieved without other sealing means, said other sealing means can nevertheless bring about an absolutely airtight seal. No absolutely airtight seal might for example result, if the used jacket is too wide in comparison with the thickness of the penis, or if the jacket has been used too often. In both cases result creases on the jacket covering the penis. Said sealing means will take out the creases. Furthermore an unfavourable shape of the hollow body (2) may prevent an absolutely airtight seal, for example if the surface of the hollow body (2) is not even enough, so that a slight permeability results.

Preferably the transmission of the tractive force takes place by a separate pulling band or belt (which is not shown as it is not part of this invention) on the pulling means attached to the hollow body (2). As the tractive force is exerted on the hollow body (2), simple pulling means can be used. Regarding the structure of the pulling means suitable for the transmission of the tractive force on the hollow body (2), this invention allows for considerable scope in its construction. For example hook, eye, clamp or clip, shackle or loop can be used for the pulling means. If pulling means are already mounted to the above-mentioned separate pulling band or belt, then only a suitable element must be attached to the hollow body (2) to serve as a pulling means (7). If for example a spring hook is mounted to the pulling band or belt, then it is sufficient to fasten a textile, plastic or metal loop to the hollow body (2), which is engaged in the spring hook. If as an alternative a clip is mounted on the pulling-belt, then preferably pulling means (7) should be attached to the hollow body (2) which can be gripped by the clip without difficulty. In the reverse case the same applies correspondingly: If for example a spring hook is mounted to the hollow body (2), then it is sufficient to fasten a loop to the separate pulling band or belt, which is engaged in the spring hook. If alternatively a clip is mounted on the hollow body (2), then preferably a pulling means should be attached to the separate pulling band or belt which can be gripped by the clip without difficulty.

In the interior cavity (3) of the hollow body (2) a vacuum (lowered pressure) is created in the moment of the introduction of a tractive force on a pulling means (7) fixed to the hermetically sealed hollow body (2). The vacuum causes a sucking force that is distributed evenly over all the areas adjoining the inner cavity (3) of the hollow body (2). As the penis (1) is introduced into the inner cavity (3) of the hollow body (2), the sucking force is consequently distributed evenly over the surface of the introduced part of the penis (1), which is exposed to the vacuum. During the treatment the penis (1) lies sideways, close to the body of the user. The length of the hollow body (2) can be adjusted to suit the individual requirements of the user. The hollow body (2) may be, along its axis, straight or curved.

Normally, rigid material should be used in the construction of the hollow body (2) so that crushing of the hollow body (2) resulting from extreme negative pressure is avoided. Whether the hollow body (2) will be deformed or even crushed does, however, also depend on its shape, as the sucking force is distributed evenly over all points of the inner cavity (3) of the hollow body (2). Consequently also elastic materials can be used. This may even be desired, so that the hollow body (2) is deformed in the moment when a certain pulling force is exceeded. The deformation could be a signal to the user to reduce the tractive force. Therefore the hollow body (2) can be made of any desired material, which can be both rigid or elastic. For example plastic, glass, rubber, metal, ceramics, wood or composite material can be used for the production of the hollow body (2). In addition, the shape of the hollow body (2) may be varied as required. For example it can be arranged so that the hollow body (2) is, in the direction of its distal closed end (2b), thickened, tapered or remains as it is. The hollow body (2) can have any desired container or vessel form, such as for example the form of a box, tin, can, bottle, cylinder, (collapsible or non-collapsible) tube or cup.

Recent research has shown that in case of penis stretching by a vacuum painless micro-fibre tears result, in which the body inserts new cells. In this way results a lengthening and thickening of the penis.

As the treatment with this invention is absolutely painless and discreet and the fastening procedure is unproblematic, its use is possible during the daily work for many hours. As furthermore the continuous stretching of a comparatively larger penis surface is achieved by this invention, a durable extension of the penis (not only of the glans) takes place already after a few months of continuous application during various hours daily. In the hollow body (2) perforations or breakpoint areas can be provided that allow the hollow body (2) to be shortened to the desired length by tearing parts of the hollow body (2) at the perforations or breakpoints.

The aforementioned solution of the problem complies with the characteristics of claim 1. Further modified versions of this design have the characteristics mentioned in the further claims, the sub-claims and the examples given for carrying out the invention.

### BRIEF DESCRIPTION OF THE DRAWINGS

The present invention is explained in greater detail below on the basis of various preferred illustrative embodiments which are represented in the drawings, in which:

FIG. 1 shows a preferred design of the penile lengthening vacuum device, comprising a hollow body (2) with a proximal open end (2a) for the admission of a penis (1), a jacket (condom (5)) with two open ends, a pulling means (7) and a Latex band (11).

FIG. 2 shows another preferred design of the penile lengthening vacuum device, comprising a hollow body (2), a jacket represented as a sleeve (6) and a pulling means (7).

FIG. 3 shows a further preferred design of the penile lengthening vacuum device, comprising a hollow body (2), a jacket (14) and a pulling means (7) attached to a screw top (10).

FIG. 4 shows a spring hook as pulling means (7).

FIGS. 5+6 show different loop designs functioning as pulling means (7).

FIG. 7 shows a penile lengthening vacuum device with a hollow body (2) tapered in direction of its distal closed end (2b), whereby the hollow body (2) has a deepened portion (2g), which enables the engaging of pulling means.

FIG. 8 shows a screw top (10) with a lateral thickening (17), which facilitates the engaging of pulling means (7).

FIG. 9 shows a screw top (10) screwed on a hollow body (2), the lower edge (18) of which enables the engaging of pulling means (7).

### DETAILED DESCRIPTION OF THE PREFERRED EMBODIMENTS

Other objects and features of the present invention will become apparent from the following detailed description, considered in conjunction with the accompanying drawing figures. It is to be understood, however, that the drawings are designed solely for the purpose of illustration and not as a definition of the limits of the invention.

Referring now to the drawings in detail, the various embodiments of the present invention will now be discussed.

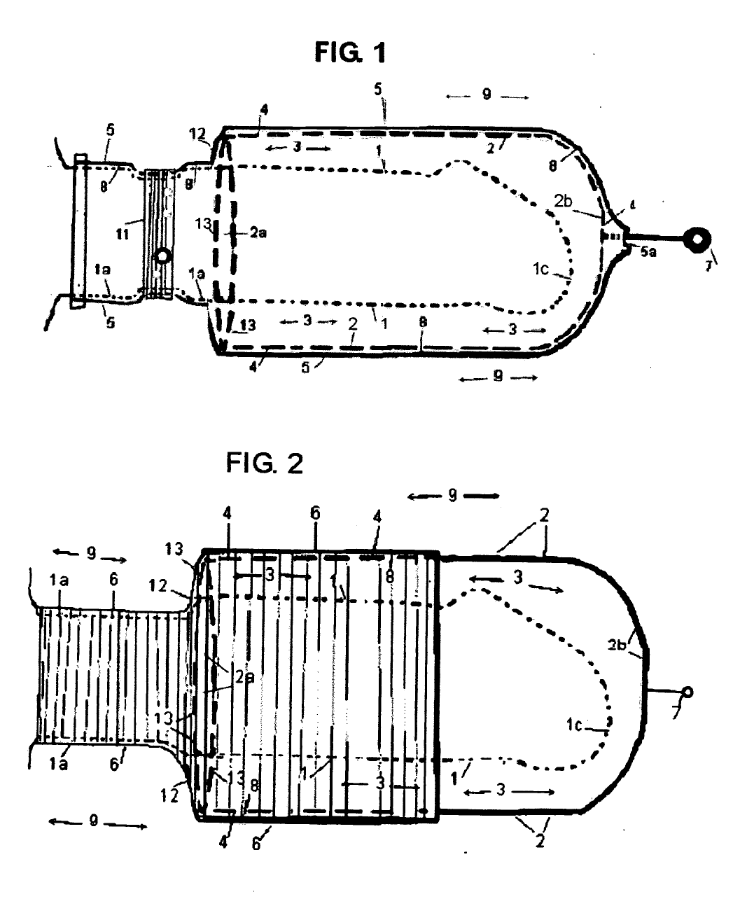

FIG. 1 shows a preferred design of the penile lengthening vacuum device, comprising a hollow body (2) with a proximal open end (2a) for the admission of a penis (1), a jacket (condom (5)) with two open ends, pulling means (7) and a Latex band (11).

The invented appliance is put into use as follows: If the jacket is—as in this case—a condom (5), then the condom (5) is cut off at its tip (5a) and the pulling means (7) (attached to the hollow body (2)) is introduced through the newly created opening in the tip (5a) of the condom (5). With the application of a moderate degree of force, the rolled up condom (5) is pulled onto the outer surface (4) of the hollow body (2), and is unrolled on it up to shortly before the proximal open end (2a) of the hollow body (2). The outer diameter of the hollow body (2) should preferably be only approx. 1-3 mm larger than the outer diameter of the thickest part of the penis. In every case the hollow body (2) (into which the penis (1) must be introduced) has an outer diameter that exceeds that of the penis (1). As a result, the compressive force of the jacket is so great that an absolutely airtight seal is achieved between the inner surface (8) of the jacket and the outer surface (4) of the hollow body (2). As a result the exterior air with atmospheric pressure (9) cannot penetrate (between inner surface (8) of the jacket and outer surface (4) of the hollow body (2)) into the interior cavity (3) of the hollow body (2). Now the penis (1) is introduced into the hollow body (2). The penis (1) should be inserted in a way that the tip (1c) of the penis (1) either touches the distal closed end (2b) of the hollow body (2), or is inserted up to a position shortly before the distal closed end (2b)—as shown on this figure. (The functioning of the invention does, however, not depend on whether the penis (1) is inserted exactly until the distal closed end (2b) of the hollow body (2), as the desired suction force is produced also if the penis (1) is not completely inserted into the hollow body). After that the condom (5) is unrolled beyond the rim of the proximal open end (2a) of the hollow body (2), and still further in the proximal direction over the proximal part (1a) of the penis (1) projecting from this end (2a). The highly elastic jacket (condom (5)) produces, as a result of its compressive action, normally an airtight seal of the inner cavity (3) of the hollow body (2), as the sealing jacket (condom (5)) presses on the skin of the proximal part (1a) of the penis (1) adjacent to the proximal open end (2a) of the hollow body (2). The jacket envelopes the proximal part (1a) of the penis (1) protruding from the hollow body (2) and makes it airtight. Consequently, on the proximal open end (2a) of the hollow body (2), the space that is not filled by the proximal part (1a) of the penis (1) is hermetically sealed. The aforementioned space between the outer rim (13) of the proximal open end (2a) of the hollow body (2) and the skin of the proximal end (1a) of the penis (1) is as a result hermetically sealed by that surface part (12) of the jacket (condom 5)), which envelopes this space. Due to this envelopment, the penetration of the exterior air with atmospheric pressure (9) into the inner cavity (3) of the hollow body (2) (along the skin of the proximal part (1a) of the penis (1)) is prevented. The condom (5) should be unrolled as far as possible over the proximal part (1a) of the penis (1) projecting from the hollow body (2), thereby sealing this part of the penis (1). The highly elastic jacket (condom 5)), as a result of its compressive force, causes an airtight closing of the inner cavity (3) of the hollow body (2), as the sealing jacket lies on the penis (1) immediately next to the proximal open end (2a) of the hollow body (2).

If a moderate continuous traction force is exerted, a standard condom (5) is fully sufficient for a number of long-lasting applications, as a large part of the traction force is—due to the vacuum resulting from this traction force—transformed into a suction force as the exterior air with atmospheric pressure is prevented from penetrating into the hollow body (2). As a result only a small portion of the traction force is not transformed into a suction force. Furthermore the inner surface (8) of the condom (5) covering the proximal part (1a) of the penis (1) (adjacent to the proximal open end (2a) of the hollow body (2)) is pressed by the suction effect against the skin of the penis (1), whilst that part of the condom (5), which covers the proximal open end (2a) of the hollow body (2), is drawn into same by the suction force. Therefore the tractive force exerted on that part of the jacket (condom (5)), which covers the proximal part (1a) of the penis (1) is considerably reduced. Therefore the jacket (condom (5)) does not tear.

Only in case much larger and very long lasting tractive forces are exerted, the use of condoms with somewhat thicker walls (which are, however, also customary in trade and therefore not expensive) should be preferred. Alternatively instead of only one, two or more standard condoms can be used, which lie over one another, and which wholly or partly overlap.

The pulling means (7) attached to the hollow body (2) must then be connected to a pulling means of a separate pulling band or belt, which is not shown as it is not covered by this invention. As a result of the introduction of a pulling force on the pulling means (7) attached to the hollow body (2), a vacuum (lowered air pressure) is created in the interior cavity (3) of the hollow body (2). As a result of the lowered air pressure a suction force is created, which has the above-mentioned positive effects. Although an airtight seal of the hollow body (2) has normally already been produced by the compressive force of the highly elastic jacket, this airtight seal can be guaranteed also for longer periods if auxiliary sealing means, such as at least one Latex band (11), any kind of rubber band or at least one piece of silicone sleeve (6) or Velcro band is lying tightly around the jacket. Said auxiliary sealing means can be spanned around both that part of the jacket, which lies close to the outer surface (4) of the hollow body (2), and around that part of the jacket, which lies close to the proximal part (1a) of the penis (1).

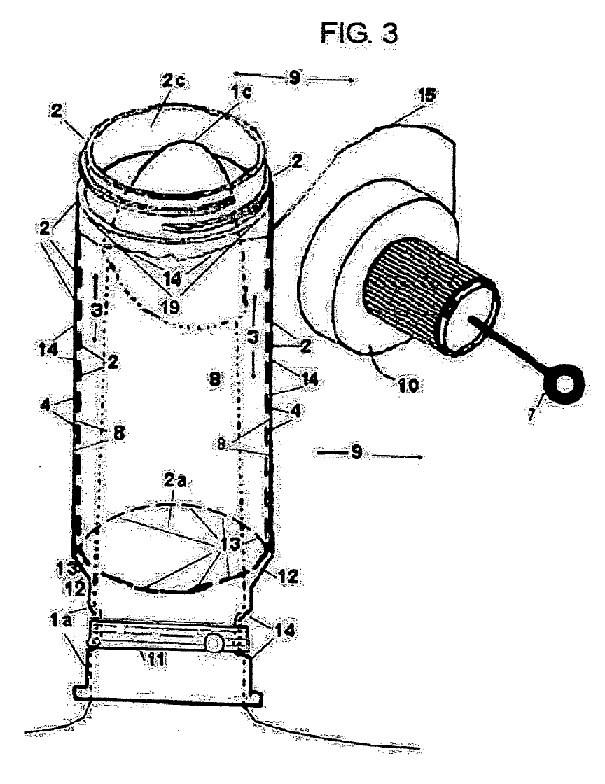

FIG. 2 shows another preferred design of the penile lengthening vacuum device, comprising a hollow body (2) with a proximal open end (2a) for the admission of a penis (1), a jacket represented as a silicon sleeve (6), customary in trade, with two open ends and a pulling means (7).

In case the jacket is a highly elastic silicon sleeve (6) with two openings (as in this figure), then first of all the penis (1) must be introduced into the hollow body (2) in a way that the tip (5a) of the penis (1) either touches the distal closed end (2b) of the hollow body (2), or is inserted up to a position shortly before the distal closed end (2b)—as shown on this figure. Subsequently the sleeve (6) is, with one of its openings, to be pulled over the pulling means (7) attached to the hollow body (2), further over the hollow body (2) itself and finally over the proximal part (1a) of the penis (1), so that the same silicone sleeve (6) hermetically envelopes a part of the outer surface (4) of the hollow body (2) until its proximal open end (2a), and the proximal part (1a) of the penis (1) jutting out from this end (2a). The absolutely airtight seal of the inner cavity (3) of the hollow body (2) is achieved as further detailed in FIG. 1. The invented device is then already fully functional. Instead of only one sleeve (6) with a large longish size, two or more shorter sleeves can be used which lie over one another, and which partly overlap. The pulling means (7) attached to the hollow body (2) has then to be connected to the pulling means of a separate pulling band or belt, which is not covered by this invention. In the interior cavity (3) of the hollow body (2) a vacuum (lowered pressure) is created in the moment of the introduction of a tractive force on the pulling means (7). The vacuum causes a sucking force that is distributed over all the areas adjoining the inner cavity (3) of the hollow body (2). As the penis (1) is introduced into the inner cavity (3) of the hollow body (2), the sucking force is consequently distributed evenly over the surface of the introduced part of the penis (1), which is exposed to the vacuum.

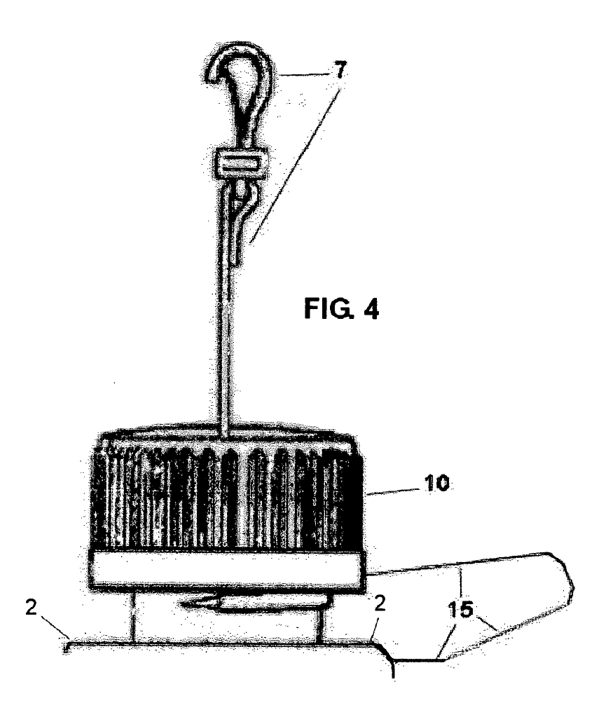

FIG. 3 shows a further preferred design of the penile lengthening vacuum device, comprising a hollow body (2) with a proximal open end (2a) for the admission of a penis (1), and with a distal open end (2c) adapted for shutting this end (2c) with a screw top (10) (screw closure), a jacket (14) with two open ends, a screw top (10) fastened to the hollow body (2) by means of a rubber band (15) and a pulling means (7) attached to the screw top (10).

The jacket (14) shown here can be both a standard condom or a silicon sleeve (both customary in trade). If the jacket (14) is a condom the invented appliance is put into use as mentioned in FIG. 1. The procedure mentioned in FIG. 2 has to be applied, if the jacket (14) is a sleeve. The only difference in both cases is that both the condom and the sleeve have to be pulled in addition over the shut closure, namely 1) over the pulling means, 2) over the shut closure and then 3) further over the hollow body (2) and finally 4) over the proximal part (1a) of the penis (1).

As already mentioned under FIG. 1, the outer diameter of the hollow body (2) should preferably be only approx. 1-3 mm larger than the outer diameter of the thickest part of the penis. The closure (10) can normally be shut before the penis is inserted into the hollow body (2). An alternative kind of application is advisable in the following case: The closure (10) should be removed during the introduction of the penis, if the outer diameter of the hollow body (2) does hardly exceed the outer diameter of the thickest part of the penis, in order to avoid that the penis compresses the air in the hollow body (2) when entering same. To enable the removal of the closure (10) the opened tip (5a) of the condom must be pulled back in the proximal direction over the closure, until the closure (10) can be opened without any problem. A sleeve would, however, normally not cover the closure. After the insertion of the penis in the hollow body (2) same has to be shut with the closure (10) (if same has not already been shut earlier). In case of this alternative both the condom and the sleeve have to be pulled over the pulling means, the shut closure and then further over the hollow body (2) until shortly before the proximal end of the hollow body. Only after the insertion of the penis, both the condom and the sleeve must be pulled further beyond the proximal end of the hollow body over the proximal part of the penis.

In order that the jacket (14) (condom or sleeve) is pressed tightly against the skin of the proximal part (1a) of the penis (1) and to guarantee an airtight seal also for a longer period, at least one Latex band (11) (or alternatively at least one piece of silicon sleeve, Velcro band or any kind of rubber band) can be spanned fast around that part of the jacket (14) which covers the proximal part (1a) of the penis. A hollow body (2) with a closure (10) has various advantages: 1) Water can be passed without any problem by simply opening the closure (10), which is connected with the hollow body (2) by a rubber band (15) or a similar connecting part. As is obvious from this FIG. 3, water can be passed without wetting the hollow body (2) or the closure (10). This the more since the hollow body (2) can be moved somewhat in the proximal direction before urinating. 2) If (after having taken off the Latex band (11)) the hollow body (2) shall be removed from the penis (1), this is easily possible after the opening of the closure (10), as this way an under-pressure is not produced when removing the hollow body (2). Such under-pressure would otherwise result in the jacket (14) being clamped fast by suction on to the penis (1). 3) After having removed the closure (10), the user can inspect at least the point of the penis (1) (glans and foreskin). In case a transparent hollow body is used, the inspection is also possible during the treatment. If there are any skin alterations the user can make a break with the use of the invention. 4) Different pulling means can be fixed to the majority of the closure types (or their break-off parts (19)). Consequently the closure (10) can for example be one of the following types:—screw, tipping, turning, bayonet, prong, clamp, stopper, pressure, snap, swinging, clip, Velcro or click-lock closure.

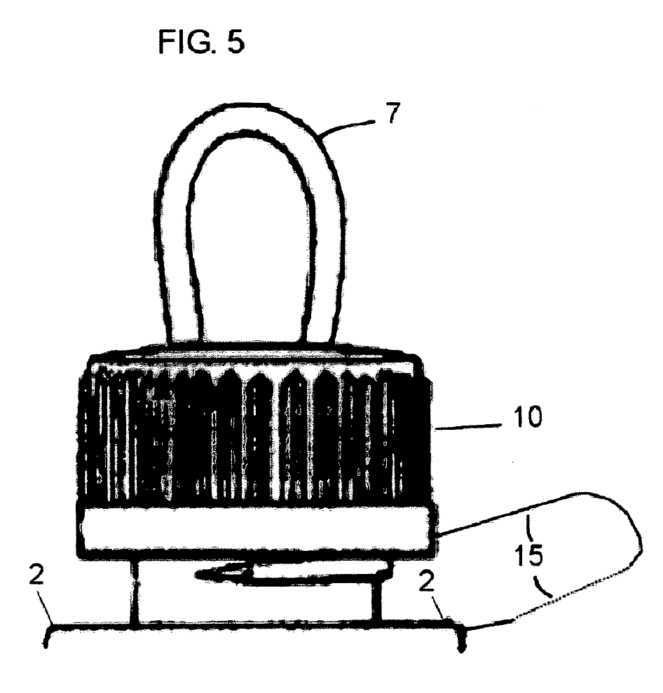

FIG. 4 shows a spring hook as pulling means (7)

A rod fastens the spring hook to the screw top (10), which in turn is fastened to the hollow body (2) with a rubber band (15), if it is screwed off. The pulling means (7) can comprise at least one item of the following:—hook, eye, clamp, clip, band, twine, thread, lace cord, string, wire, cable, cord, loop, ring, shackle, tong or Velcro band.

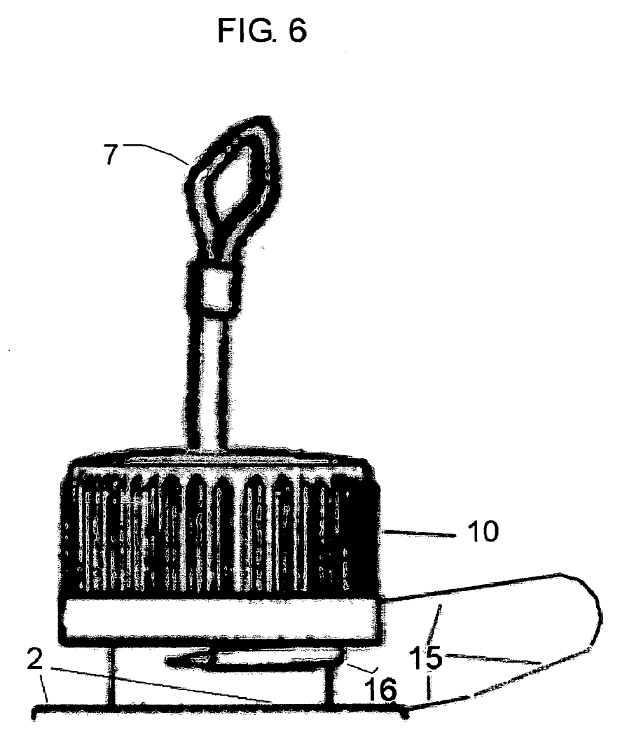

FIGS. 5+6 show different loop designs functioning as pulling means (7).

The loop in FIG. 5 is directly fixed to the screw top (10), whilst both ends of the loop in FIG. 6 are fastened to a pulling band, which in turn is fastened to the screw top (10). The pulling means shown as examples in FIGS. 4-6 can either be fastened by any desired fastening means to a closure (10) of any desired type, or to the hollow body (2). Said pulling means are preferably fastened to the outer surface (4) of the distal closed end (2b) of the hollow body (2). Alternatively the fastening is, however, also possible to the lateral outer surface, to the surface of the outer rim (13) of the proximal open end or to the inner surface of the hollow body (2). It is—for example—also possible to fasten a part of a pulling means (7) (e.g. a loop) to the screw thread (16) of the screw top (10). The fastening can also be carried out merely by shutting the closure (10), namely if during the shutting procedure the part of a pulling means is clamped. The pressing force caused by the shutting procedure thereby enables the fastening of at least one part of a pulling means (which is either covered by this invention or belonging to a separate pulling band or belt not covered by this invention) to the screw top (10) or any other type of closure.

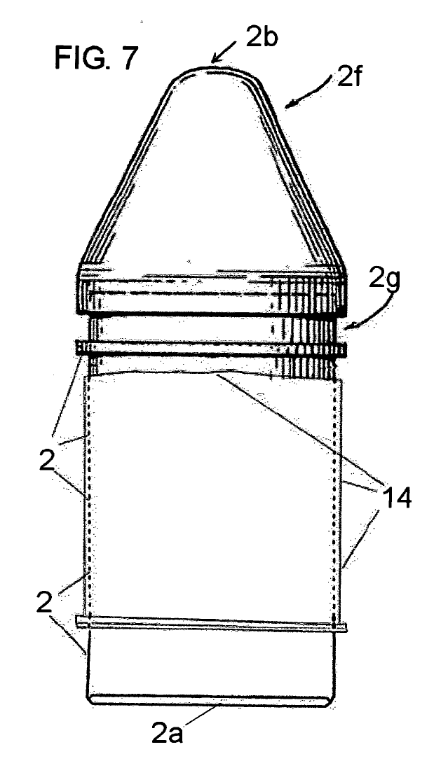

FIG. 7 shows a penile lengthening vacuum device with a hollow body (2) tapered in direction of its distal closed end (2b), whereby the hollow body (2) has a deepened portion (2g), which enables the engaging of pulling means.

The tapered hollow body (2) has a deepened portion (2g) behind its point (2f), which resembles the point of a penis (glans) (1). A pulling means of the separate pulling band or belt, which is not covered by this invention, can engage in this deepened portion (2g). This pulling means can for example be designed as a pincer-shaped device or a clicking into place-device. Consequently the shown hollow body (2) does not require own pulling means. Consequently a pulling means attached to the hollow body (2) is not necessary. The hollow body (2) is covered by a jacket (14) (being in this figure a condom). The condom is unrolled until shortly before the proximal open end (2a) of the hollow body (2). The hollow body (2) is this way ready for the introduction of a penis (1) into the proximal open end (2a).

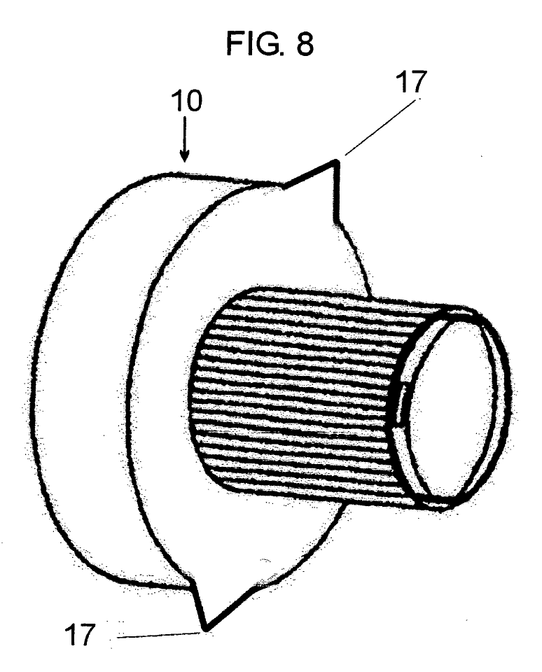

FIG. 8 shows a screw top (10) with a lateral thickening (17), which enables the engaging of a pulling means (7).

The hollow body (2) or the closure (10) can be designed in a way, that a traction force can be exerted on them without that a pulling means (7) is mounted on them. The exertion of a tractive force can be enabled by the existence of structures on the walls of the hollow body (2) or the closure (10) or on a break-off part ((19) in FIG. 3)). The aforementioned structures can be gripped, for example, by a separate device attached to the pulling band or belt (not covered in the specification of this invention), such as a pincer-shaped device, a clip, a snap or a click-lock type device, or these devices can engage in said structures. As an example, such special structures can be: at least one thickening (17), deepening (2g), opening, plate, thread or edge. As is known, a hollow body (2) or closure (10) can be provided with a—preferably annular—break-off part ((19) in FIG. 3) having a predetermined breaking point. When the closure (10) is opened the first time, this break-off part (19) breaks off. This part (19) is normally ring-shaped and therefore remains fixed to the hollow body (2) or closure (10). The broken off part (19) normally signals that a container or vessel has been opened, and it has no other function. Now, however, this part (19) can serve for the fastening of either the pulling means (7) of this invention, or pulling means of the separate pulling band or belt which is not covered by this invention. For example at least one hole can be created in the annular break-off part (19) through which for example a wire (which belongs to the pulling means) is lead.

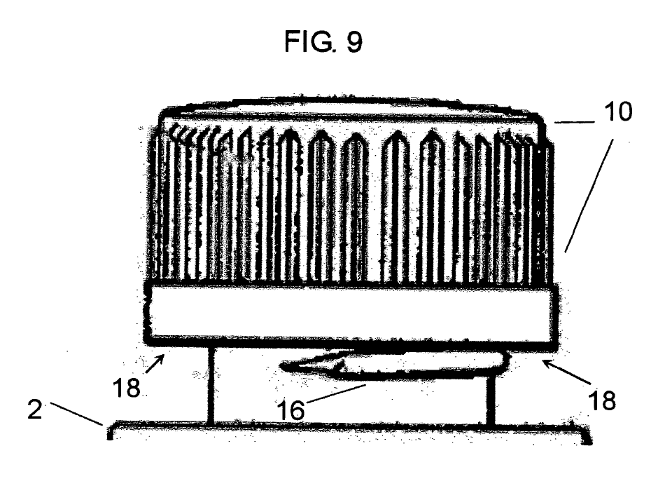

FIG. 9 shows a screw top (10) screwed on a hollow body (2), the lower edge (18) of which enables the engaging of pulling means (7).

On the lower edge (18) of a screw top (10) a tractive force can be exerted (by one of the devices detailed in FIG. 8) after screwing the top tight to the hollow body (2). The lower edge (18) is shaped in a way that sufficient distance is allowed from a hollow body (2) provided with a screw winding (16), so that the engaging by one of the devices detailed in FIG. 8 is enabled. Alternatively the fastening can take place merely as the result of the pressure exerted by screwing the screw top (10) tight (or by shutting any other kind of closure).

APPENDIX
LIST OF DRAWING REFERENCES
The following table contains the reference numbers used in the
attached drawings.

1 = penis
1a = proximal part of the penis protruding from the hollow
body
1c = penis point(point of glans penis)
2 = hollow body
2a = proximal open end of the hollow body for the admission of
the penis
2b = distal closed end of the hollow body
2c = distal open end of the hollow body adapted for the
attachment of a closure (10)
2f = point of the hollow body
2g = deepening
3 = inner cavity of the hollow body
4 = outer surface of the hollow body
5 = condom
5a = opening at the tip of the condom
6 = sleeve
7 = pulling means
8 = inner surface of the jacket (14) (condom (5) or sleeve
(6))
9 = exterior air with athmospheric pressure
10 = screw top (closure)
11 = Latex band
12 = surface part of the jacket (14), which envelopes the
space between the outer rim (13) of the proximal open end (2a)
of the hollow body (2) and the skin of the proximal part (1a)
of the penis (1), thereby hermetically sealing this space
13 = outer rim of the proximal open end (2a) of the hollow body
(2)
14 = jacket
15 = rubber band
16 = screw winding
17 = thickening
18 = lower edge
19 = break off part

What is claimed is:

1. A wearable penile lengthening vacuum device comprising:
a hollow body having a first proximal open end, a second distal closed end and an inner cavity, said proximal open end and said inner cavity being adapted for the introduction of a penis;
a highly elastic jacket having two open ends, whereby a proximal part of the jacket extends continuously around the skin of the proximal part of the introduced penis protruding from the hollow body, and a distal part of the same jacket extends continuously around the outer surface of said hollow body;
pulling means attached to the hollow body;
and wherein said jacket is sized for tightly wrapping said proximal part of the penis and said outer surface of the hollow body, and has an elasticity sufficient for the airtight or almost airtight sealing of the inner cavity of the hollow body.

2. A device according to claim 1, wherein said pulling means is fastened to one or more surfaces of the hollow body selected from the group consisting of the outer surface of the distal closed end, the lateral outer surface, the surface of the outer rim of the proximal open end, the inner surface and any combination thereof.

3. A device according to claim 1, wherein said pulling means is formed from one or more materials selected from the group consisting of hook, eye, clamp, clip, band, twine, thread, string, wire, cable, cord, loop, ring, shackle, tong, Velcro band and any combinations thereof.

4. A device according to claim 1, wherein the hollow body is manufactured from one or more materials selected from the group consisting of plastic, glass, rubber, metal, ceramic, wood, elastic material, rigid material and any combination thereof.

5. A device according to claim 1, wherein the type of the section of the hollow body is selected from the group consisting of straight longitudinal section, bent longitudinal section, round cross section, triangular cross section, rectangular cross section, polygonal cross section and any combination thereof.

6. A device according to claim 1, wherein the type of the diameter of the hollow body from its proximal open end to its distal closed end is selected from the group consisting of increasing diameter, decreasing diameter, unchanged diameter and any combination thereof.

7. A device according to claim 1, wherein the form of the hollow body is selected from the group consisting of box form, tin form, can form, bottle form, cylinder form, tube form and cup form.

8. A device according to claim 1, wherein the hollow body has perforations or breakpoint areas allowing the hollow body to be shortened to the desired length.

9. A device according to claim 1, wherein the type of the jacket is selected from the group consisting of a condom cut off at its tip, at least two condoms cut off at their tip and superimposed one upon another, silicone sleeve, at least two silicone sleeves superimposed one upon another.

10. A device according to claim 1, wherein the jacket is covered by one or more types of sealing means selected from the group consisting of Latex band, rubber band, Velcro band, silicone sleeve and any combination thereof.

11. A wearable penile lengthening vacuum device comprising:
a hollow body having a first proximal open end and an inner cavity adapted for the introduction of a penis and a second distal open end designed for the fastening of a closure;
a closure designed for shutting the second distal open end of said hollow body;
a highly elastic jacket having two open ends, whereby a proximal part of the jacket extends continuously around the skin of the proximal part of the introduced penis protruding from the hollow body, and a distal part of the same jacket extends continuously around the outer surface of said hollow body;
pulling means attached to the hollow body or to the closure;
and wherein said jacket is sized for tightly wrapping said proximal part of the penis and said outer surface of the hollow body, and has an elasticity sufficient for the airtight or almost airtight sealing of the inner cavity of the hollow body.

12. A device according to claim 11, wherein said pulling means is formed from one or more materials selected from the group consisting of hook, eye, clamp, clip, band, twine, thread, string, wire, cable, cord, loop, ring, shackle, tong, Velcro band and any combinations thereof.

13. A device according to claim 11, including a ring-shaped part attached to said hollow body or said closure and designed to break off from said hollow body or said closure when said closure is opened;
and wherein said pulling means is fastened to said ring-shaped part.

14. A device according to claim 11, wherein the design of the closure is selected from the group consisting of screw closure, tipping closure, turning closure, bayonet closure, prong closure, clamp closure, pressure closure, snap closure, swinging closure, clip closure, Velcro closure, click-lock closure and any combination thereof.

15. A device according to claim 11, wherein the hollow body is manufactured from one or more materials selected from the group consisting of plastic, glass, rubber, metal, ceramic, wood, elastic material, rigid material and any combination thereof.

16. A device according to claim 11, wherein the type of the section of the hollow body is selected from the group consisting of straight longitudinal section, bent longitudinal section, round cross section, triangular cross section, rectangular cross section, polygonal cross section and any combination thereof.

17. A device according to claim 11, wherein the type of the diameter of the hollow body from its proximal to its distal end is selected from the group consisting of increasing diameter, decreasing diameter, unchanged diameter and any combination thereof

18. A device according to claim 11, wherein the hollow body has perforations or breakpoint areas allowing the hollow body (2) to be shortened to the desired length.

19. A device according to claim 11, wherein the type of the jacket is selected from the group consisting of a condom cut off at its tip, at least two condoms cut off at their tip and superimposed one upon another, silicone sleeve, at least two silicone sleeves superimposed one upon another.

20. A device according to claim 11, wherein the jacket is covered by one or more types of sealing means selected from the group consisting of Latex band, rubber band, Velcro band, silicone sleeve and any combination thereof.

21. A device according to claim 11, wherein the closure is also designed for allowing the engaging of pulling means.

22. A wearable penile lengthening vacuum device comprising:
a hollow body having a first proximal open end, a second distal closed end and an inner cavity, said proximal open end and said inner cavity being adapted for the introduction of a penis;
a highly elastic jacket having two open ends, whereby a proximal part of the jacket extends continuously around the skin of the proximal part of the penis protruding from the hollow body, and a distal part of the same jacket extends continuously around the outer surface of said hollow body;
at least one structure on the lateral surface of the hollow body designed to enable the engaging of pulling means;
and wherein said jacket is sized for tightly wrapping said proximal part of the penis and said outer surface of the hollow body, and has an elasticity sufficient for the airtight or almost airtight sealing of the inner cavity of the hollow body.

23. A device according to claim 22, wherein the type of structure is selected from the group consisting of thickening, deepening, opening, plate, thread or prominent edge.
